(19) United States
(12) Patent Application Publication
(10) Pub. No.: US 2003/0082102 A1
(43) Pub. Date: May 1, 2003

(54) Radioactive platinum complexes for cancer treatment

(76) Inventor: Wayne S. Court, Cold Spring Harbor, NY (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/178,806

(22) Filed: Jun. 24, 2002

Related U.S. Application Data
(60) Provisional application No. 60/300,704, filed on Jun. 25, 2001.
(60) Provisional application No. 60/300,677, filed on Jun. 25, 2001.

(51) Int. Cl. A61K 51/00

(52) U.S. Cl. 424/1.11; 424/1.65

(57) ABSTRACT

Novel therapeutic use of stable radioisotopic forms of platinum antitumor complexes are provided. Such radioactive forms of platinum-based drugs should enhance their tumor killing ability compared to non-radioactive forms currently available and offer therapeutic alternatives to these promising drugs.

### SUMMARY OF INVENTION

This invention relates to a method of enhancing the tumor-killing ability of platinum-based drugs by producing radioisotopic (¹⁹¹Pt, ^{193m}Pt and ^{195m}Pt) forms of Carboplatin, JM216 and Iproplatin. By making these platinum-based drugs directly radioactive through their platinum moiety, it is believed that one could achieve improved therapeutic gain compared to the parent compound, Cisplatinum. Administration of these drugs will also be done by intra-arterial, intra-peritoneal, intra-thecal or intra-tumoral routes, allowing immediate tumor contact, first pass kinetics, first pass uptake and/or first pass extraction of the radioactive compounds, thereby reducing systemic radiation exposure. Finally, used in conjunction with agents to reduce systemic toxicity, such agents may provide alternative treatments for situations of tumor resistance or intolerance to cisplatinum.

The therapeutic use of nine isotopically-labeled drugs are claimed by this invention:

(a) ¹⁹¹Pt-labeled 1,1-Cyclobutanedicarboxylate diamine platinum (II)

(b) ^{193m}Pt-labeled 1,1-Cyclobutanedicarboxylate diamine platinum (II)

(c) ^{195m}Pt-labeled 1,1-Cyclobutanedicarboxylate diamine platinum (II)

(d) ¹⁹¹Pt-labeled Cis-dichloro,trans-dihydroxybis-isopropylamine platinum (IV)

(e) ^{193m}Pt-labeled Cis-dichloro,trans-dihydroxybis-isopropylamine platinum (IV)

(f) ^{195m}Pt-labeled Cis-dichloro,trans-dihydroxybis-isopropylamine platinum (IV)

(g) ¹⁹¹Pt-labeled bis-acetato-ammine-dicholoro-cyclohexylamine platinum (IV)

(h) ^{193m}Pt-labeled bis-acetato-ammine-dicholoro-cyclohexylamine platinum (IV)

(i) ^{195m}Pt-labeled bis-acetato-ammine-dicholoro-cyclohexylamine platinum (IV)

### BACKGROUND OF INVENTION

The rationale behind this application is based on several pertinent observations:

(1) Platinum-based drugs are in common clinical use as chemotherapy for a variety of malignant tumors.

The introduction of Cisplatinum in the first generation of platinum-based drugs brought safe and effective treatment of testicular and ovarian cancer. As clinical experience evolved, Cisplatinum has shown activity against a wide variety of malignancies, as have second-generation drugs such as Carboplatin (Reed 1993).

(2) One property of Cisplatinum is the ability to enhance the tumor-killing capacity of radiation (radiosensitization).

In addition to its intrinsic tumor-killing ability, Cisplatinum has been shown to work in synergy with external radiation. Thus, the use of Cisplatinum in combination with external radiation provides greater levels of tumor killing than can be achieved by either modality alone (Barot 1985; Reed 1993).

(3) In order to take advantage of the radiation-enhancing effects of Cisplatinum, it was proposed to use a radioactive form of cisplatinum as cancer therapy.

In a separate patent the therapeutic use of ^{195m}Cisplatinum was described in detail (Order 1999). The premise of that application is that making the platinum-based drug directly radioactive should enhance the tumor-killing ability of the parent, non-radioactive compound. Clinical trials have been designed to examine the applicability of this novel therapeutic compound.

(4) As the role of Cisplatinum in cancer therapy evolved, new platinum-based compounds have been added to the oncologists' arsenal.

Although Cisplatinum has entered widespread use in the treatment of solid tumors, it often produces significant toxicity. As a partial list, use of Cisplatinum may cause damage to the kidneys, gastrointestinal tract, hearing and peripheral nerves.

The development of Cisplatinum analogues have centered upon identifying compounds with less toxicity and with a different spectrum of activity (Judson 2000; O'Dwyer 2000). Table 1 provides a partial listing of recently-developed platinum-based complexes. Cited in reference to the present invention are Carboplatin, Iproplatin and JM216.

(5) Compared to Cisplatinum, preclinical and early clinical studies have suggested that these newer platinum-based compounds are less toxic analogues.

One of the best studied 2^{nd} generation platinum-based compound is Carboplatin (Bunn 1990). In comparison to Cisplatinum, Carboplatin has proven far less toxic to kidney and nervous system and causes less nausea and vomiting, while usually retaining equivalent tumoricidal activity. Quite often, Carboplatin is becoming the drug of choice in light of the improved quality of life it provides patients. Carboplatin has therein enhanced safety while maintaining effectiveness against a variety of tumors (Reed 1993; Fischer 1997).

Another platinum-based compound, Iproplatin (Bramwell 1985; Chawla 1988; Ribaud 1986; Trask 1991) is undergoing clinical development. Early data suggests that it may prove superior to cisplatin in some therapeutic situations as they have greater efficacy against certain tumors while maintaining a relatively mild toxicity profile.

The first orally available platinum-based drug, JM216, has progressed beyond animal models, entering clinical trials in 1992 and now undergoing phase III evaluation (Kelland 2000; Kurata 2000; McKeage 1995; Sessa 1998). It has a relatively mild toxicity profile with myelosuppression being dose-limiting. In addition to the oral route, a preclinical study suggests rectal administration of JM216 may be feasible (Tanaka 1999). By providing alternate routes of administration, this drug may therein broaden the applicability of platinum-based therapy.

Of particular relevance to this invention, several of these drugs are believed to be capable of overcoming intrinsic or acquired resistance to Cisplatinum (Rixe 1996; Holford 2000). This latter feature may allow treatment of solid tumors in situations where Cisplatinum could not be used (i.e., risk of kidney damage) or in situations where it is no longer effective (drug resistance).

Table 2 overviews the clinical development and applicability of these selected compounds.

It is believed that ongoing developments in platinum-based drug therapy should translate into significant improvements in treatment for patients with a broad range of malignant tumors.

Table 3 provides an overview of current dose regimens for the non-radioactive platinum-based drugs. These dosing regimens, along with preclinical toxicology studies, provided a basis for using the proposed radioactive counterparts (Clark 1999; O'Dwyer 2000).

(6) These newer platinum-based compounds can be made radioactive at the platinum moiety.

Table 4 reveals that several of these platinum-based compounds have been radioactively labeled at the platinum moiety. To date, these radiolabeled compounds have only been used in diagnostic quantities to study drug biodistribution and pharmacokinetics.

For example, using the ^{191/193m}Pt-forms of cisplatinum, carboplatin and Iproplatin, Thatcher (1982) and Sharma (1983) studied the blood clearance of drug in patients with malignant disease. Harrison (1983) compared and contrasted the distribution of similar platinum-labeled compounds in rats. In 1985, Owens et al reported on the in vivo distribution of radioactively labeled platinum complexes using a gamma camera. The use of radiolabeled platinum analogues has been proposed as a means to non-invasively measure the tumor pharmacokinetics of drug uptake (Dowell 2000). Finally, Bates (1997) and colleagues synthesized ¹⁹¹Pt-labeled JM216 in order to perform quantitative diagnostic studies of this oral agent.

(7) The above mentioned platinum-based compounds appear to retain the radiation-enhancing properties of Cisplatinum while offering reduced toxicity and increased applicability.

There is preclinical evidence that these newer platinum compounds have radiation-enhancing properties. Shortly after its synthesis, Douple (1985) suggested that Carboplatin is a potentiator of external radiation therapy. The interaction of Cisplatin, Carboplatin and Iproplatin with external radiation has been studied in tissue culture (Skov 1991).

Howell (1994) and colleagues showed radiopotentiation of ^{195m}Pt-trans-cisplatinum in cell culture and extrapolated these observations to consider therapeutic use. In their hands, cis-^{195m}Pt showed no potentiation of chemotherapy effectiveness in cell culture, which may be attributed to a low specific activity (i.e., how radioactive the drug was made). However, these in vitro data on radiolabeled trans-cisplatinum does not support therapeutic potential. As the authors admit in their article “. . . trans-Pt may not be the ideal carrier for radioplatinum in that it is not among the select group of therapeutically effective platinum-coordination compounds.”

Areberg (2000) examined the in vitro toxicity of ¹⁹¹Pt-labeled cisplatin on a human cervical carcinoma cell line. This latter group recently extended their observations to use of ¹⁹¹Pt-cisplatin in tumor-bearing nude mice (Areberg 2001). Last, in vitro studies by Amorino (1999) suggests that radiopotentiation by JM216 is effected through inhibition of sublethal and potentially lethal damage repair.

(8) It is not known which isotopic form of these platinum-based drugs would be the preferred choice for cancer therapy.

In comparison to the ¹⁹¹Pt form, the ^{193m}/^{195m}Pt isotopes provide relatively more low-energy electrons, as well as conversion electrons, with fewer photons. However, it is not yet known whether sufficiently high specific activities (i.e., mCi/mg Pt) of these ^{193m}Pt/^{195m}Pt-labeled drugs can be made. (Areberg 2000). In this regard, using cis-^{195m}Pt for cell culture studies, Howell et al (1994) saw no radiotoxicity above and beyond its chemical toxicity (supra vide).

(9) Systemic delivery of radiolabeled platinum compounds should include other agents designed to minimize systemic exposure and resulting toxicity.

Based on prior biodistribution and pharmacokinetic studies using trace doses, radiolabeled platinum drugs given by the intravenous or oral routes may result in significant exposure of normal tissues and organs to radiation. Such exposure of normal tissues to radiolabeled compounds may result in an increase in the risk as well as severity of toxic events. Approaches to minimizing the systemic toxicity of chemotherapy that related in the present invention include (1) liposomal encapsulation (2) sodium thiosulfate and (3) Amifostine (WR2721).

It is an additional claim of this invention that the systemic administration of radiolabeled platinum compounds for therapeutic purposes should include other agents to attenuate systemic toxicity. (10) Increased tumor uptake of radioactively-labeled platinum drug can be achieved by selecting the route of administration.

Court (2001) described increased tumor remission using intra-arterial delivery of cisplatinum in patients with nonresectable hepatoma. Using tracer quantities of ^{195m}Pt-cisplatinum, it was demonstrated that intra-arterial infusion of cisplatinum selectively exposes the tumor to higher drug levels (i.e., 34-55% of given dose) than can be achieved by the intravenous route (<5% of given dose). This selective tumor uptake demonstrated first-pass kinetics.

It is believed that increased tumor uptake of radioactive platinum-based drugs can be achieved by administration via intra-arterial, intra-peritoneal, intra-thecal or intra-tumoral routes. The rationale for increased tumor uptake can be explained by immediate tumor contact and binding, first pass kinetics, first pass uptake and/or first pass extraction of the radioactive compounds, thereby reducing systemic radiation exposure and potential toxicity.

Synopsis of Therapeutic Drug Use:

Title: ¹⁹¹ Pt, ^{193m}Pt, and ^{195m}Pt-labeled Carboplatin, Iproplatin and JM216 in the treatment of solid tumors

Duration of Treatment: Maximum 12 months

Inclusion Criteria:

Tissue proof of malignant tumor is required.

Solid tumors may include primary cancers of the ovary, bladder, brain, breast, testes, liver, lung, cervix, endometrium, colorectum, head and neck.

WBC 3,000 cells/cc or greater.

Platelets 140,000 cells/cc or greater.

Hemoglobin may be transfused to 9 or greater.

Performance status of ≧70% Karnofsky scale.

Creatinine 1.5 mg/dL or less.

BUN 25 mg/dL or less.

If at risk, pregnancy test must be performed. If positive, not eligible.

Exclusion Criteria:

Absence of any of the inclusion criteria

Ascites, malignant or non-malignant

Portal venous occlusion

Hepatic renal syndrome

Hypercalcemia (>10 mg/dL)

Hyperglycemia (>200 mg/dL)

Hypoglycemia (<60 mg/dL)

Pregnancy (if at risk, pregnancy test must be performed)

Drugs: ¹⁹¹ Pt, ^{193m}Pt and ^{195m}Pt-labeled and the non-radioactive forms of Carboplatinum, Iproplatin and JM216

Drug Administration:

Carboplatin is administered as a solution in normal saline or 5% dextrose solution over 15 to 30 minutes. Iproplatin is given in one liter normal saline over one hour. JM216 is given orally in gelatin capsules.

As filler for injection use, sugar solutions, buffer solutions, ethylene glycol, polyethylene glycol and the like may be used.

As fillers, diluents and auxiliaries of oral administration preparations, one or more materials can be used which may be selected from lactose, sucrose, glucose, sorbitol, mannitol, potato starch, amylopectin, other various starches, cellulose derivatives (for example, carboxymethylcellulose, hydroxyethyl cellulose and the like), gelatin, magnesium stearate, polyvinyl alcohol, calcium stearate, polyethylene glycol, gum arabic, talc, titanium dioxide, vegetable oils such as olive oil, peanut oil, sesame oil and the like, paraffin oils, neutral fat bases, ethanol, propylene glycol, physiological saline, sterile water, glycerol and the like.

Dosing Regimen: The treatment of solid tumors with these drugs are believed to be therapeutically effective if given in the following range of dosages for the specified time periods.

Overview of Drug Dosing and Delivery:

The total dose of each drug will contain various ratios of standard nonradioactive drug and its radioactively-labeled analogue.

Both standard platinum-based drug and the radioactively-labeled drug solution will contain 1 mg of drug per ml.

191 Pt, ^{193m}Ptand ^{195m}Pt-labeled drugs will contain 1 mCi/mg (solution will contain 1 mCi/ml).

∘ A diagnostic dose of 1 mCi of radiolabeled drug will be injected

∘ SPECT/planar imaging and dosimetry will be performed for four days to determine caluclated uptake by organs of interest

∘ The remaining dose of radiolabeled drug will be infused or

∘ Based on the calculated uptake by normal organs of interest, the total dose of radiolabeled drug will not exceed the following limits: liver: ≧600 cGy, Kidney: ≧300 cGy, Marrow ≧40 cGy.

∘ The remaining standard (nonradioactive) drug will be infused to bring the total dose of hot+cold drug to following levels (see table above): Carboplatin 360 mg/m2; Iproplatin 300 mg/m2; JM216 100 mg/m2.

¹⁹¹Pt, ^{193m}Pt and ^{195m}Pt-labeled Carboplatin or Iproplatin will be given as an intravenous, intraarterial, intraperitoneal, or intra-tumoral infusion followed one to two hours later by an infusion of the remaining amount of nonradioactive drug.

¹⁹¹Pt ^{193m}Pt and ^{195m}Pt-labeled JM216 will be given orally in a gelatin capsule followed one to two hours later by an oral capsule of the remaining amount of nonradioactive JM216.

Dosing Regimen for Carboplatinum:

The total dose of Carboplatinum will contain 5 to 50 mg/m² of radioactively labeled Carboplatinum analogues along with the standard drug according to the following schedule (assuming a 1 m² patient):

| ¹⁹¹Pt, ^{193m}Pt or | | | |
|---|---|---|---|
| ^{195m}Pt- | Standard | | |
| Carboplatin | Carboplatinum | Total Carboplatin | Total Radioactivity |
| (mg) | (mg) | (mg) | (mCi) |
| 5 | 355 | 360 | 5 |
| 10 | 350 | 360 | 10 |
| 20 | 340 | 360 | 20 |
| 30 | 330 | 360 | 30 |
| 40 | 320 | 360 | 40 |
| 50 | 310 | 360 | 50 |

Accordingly, a total dose of 360 mg/m² of carboplatin will be administered every 4 weeks

Dosing Regimen for JM216: ∘

The total dose of JM216 will contain 5 to 50 mg/m² of radioactively labeled JM216 analogues along with the standard drug according to the following schedule (assuming a 1 m² patient):

| ¹⁹¹Pt, ^{193 m}Pt or | | | |
|---|---|---|---|
| ^{195 m}Pt-JM216 | Standard JM216 | Total JM216 | Total Radioactivity |
| mg | (mg) | (mg) | (mCi) |
| 5 | 95 | 100 | 5 |
| 10 | 90 | 100 | 10 |
| 20 | 80 | 100 | 20 |
| 30 | 70 | 100 | 30 |
| 40 | 60 | 100 | 40 |
| 50 | 50 | 100 | 50 |

Accordingly, a total dose of 100 mg/m² of JM216 will be administered orally for 5 days every 5 weeks

Dosing Regimen for Iproplatin:

The total dose of Iproplatinum will contain 5 to 50 mg/m² of radioactively labeled Iproplatinum analogues along with the standard drug according to the following schedule (assuming a 1 m² patient):

| ¹⁹¹Pt, ^{193m}Pt or | Standard | | |
|---|---|---|---|
| ^{195m}Pt-Iproplatin | Iproplatinum | Total Iproplatin | Total Radioactivity |
| (mg) | (mg) | (mg) | (mCi) |
| 5 | 295 | 300 | 5 |
| 10 | 290 | 300 | 10 |
| 20 | 280 | 300 | 20 |
| 30 | 270 | 300 | 30 |
| 40 | 260 | 300 | 40 |
| 50 | 250 | 300 | 50 |

Accordingly, a total dose of 300 mg/m² of iproplatin will be adminstered every 2 weeks

Dosing Rules:

A diagnostic dose of 1 mCi of radiolabeled drug will be injected

SPECT scanning and dosimetry will be performed for four days to determine calculated uptake by organs of interest

The remaining dose of radiolabeled drug will be infused or:

Based on the calculated uptake by normal organs of interest, the total dose of readiolabeled drug will not exceed the following limits: liver: ≧600 cGy, Kidney: ≧300 cGy, Marrow ≧40 cGy.

The remaining standard (nonradioactive) drug will be infused to bring the total does of hot+cold drug to the levels described in the above tables.

Dose escalation is stopped if 2 of 3 patients in a dose group develop Grade 3 or greater renal, audiometry or hematological or other toxicity according to the National Cancer Institute Common Toxicity Criteria.

Patients who develop mild toxicity, Grade 1-2 and recover, may continue on their assigned monthly dose.

This procedure is repeated until all groups have been dosed.

Patients may remain on the same dose of drug for up to one year, if clinically indicated.

Dosing and Termination Rules:

Patients will receive the same dose every interval.

Before the third dose, if tumor volumetrics or if the biochemical tumor marker titer increases greater than 25%, the patient will be removed from the study and no longer receive the test drug.

If partial remission occurs, the same dose will be administered at the designated intervals until progression, complete remission or toxicity occurs.

Response Criteria:

∘ Complete remission (CR): disappearance of all clinical evidence of tumor for a minimum of one month

∘ Partial remission(PR): ≦50% decrease in the volume of all measurable lesions on contrast-enhanced CT scan, or a similar decrease in tumor marker titer

∘ Stable disease (SD): <50% reduction or ≦25% increase in tumor volume or tumor marker titer

∘ Progressive disease (PD): >25% increase in tumor volume of all measurable lesions on contrast-enhanced CT scan or by tumor marker titer

Drug Assessments: A schematic of clinical assessments during the period of drug delivery is provided below:

### Pretreatment (Screening) Visit: Visit 0

At the pretreatment visit, the following will be performed:

Informed Consent

Complete history and physical

CT or MRI of involved organs. Note: In some patients remission begins at one month. Tumor volumes can be calculated at that time compared to the original tumor volume. Remission must be documented before a third cycle of drug is infused either by tumor volumetics or 50% reduction of tumor marker or both¹.

CBC, platelets, BUN, creatinine, liver chemistries.

EKG.

Audiometry

Tumor markers

### Visit 1: Initial Treatment Visit

At Visit 1 the following will be performed:

CBC, platelets

Signs and Symptoms

CT or MRI

Schedule of Assessments

| Visit | 0 | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|---|
| Week (W) | | W0 | W1 | W2 | W3 | W4 | | | | | | | | | | | |
| Month (M) | | | | | | M1 | M2 | M3 | M4 | M5 | M6 | M7 | M8 | M9 | M10 | M11 | M12 |
| Consent | X | | | | | | | | | | | | | | | | |
| History | X | | | | | X | X | X | X | X | X | X | X | X | X | X | X |
| Physical | X | | | | | X | X | X | X | X | X | X | X | X | X | X | X |
| CBC w/ | X | X | X | X | X | X | X | X | X | X | X | X | X | X | X | X | X |
| Platelets | | | | | | | | | | | | | | | | | |
| Symptoms | X | X | X | X | X | X | X | X | X | X | X | X | X | X | X | X | X |
| CT or MRI | X | X | | | | X | X | X | X | X | X | X | X | X | X | X | X |
| BUN & | X | X | X | X | X | X | X | X | X | X | X | X | X | X | X | X | X |
| Creatinine, | | | | | | | | | | | | | | | | | |
| 24 hr | | | | | | | | | | | | | | | | | |
| clearance | | | | | | | | | | | | | | | | | |
| Tumor marker | X | | | | | X | X | X | X | X | X | X | X | X | X | X | X |
| Liver Function | X | | | | | X | X | X | X | X | X | X | X | X | X | X | X |
| Audiometry | X | | | | | X | X | X | X | X | X | X | X | X | X | X | X |
| ECG | X | | | | | X | X | X | X | X | X | X | X | X | X | X | X |
| SPECT Scan | | X | | | | X | X | X | X | X | X | X | X | X | X | X | X |
| Dosing | | X | | | | X | X | X | X | X | X | X | X | X | X | X | X |

Visit 0: Screening Visit
Visit 1: Initial Dosing Visit
BUN and Creatinine
SPECT/planar imaging
Initial Dose

### Visits 2-5 (weeks 1-4)

Patients will return weekly after the first dose to evaluate safety and toxicity. The following will be performed:

CBC with platelets

Signs and Symptoms

CT or MRI (at week 4)

BUN and creatinine

Audiometry

### Visits 6-16 (Months 2 to 12)

If patients do not exceed normal organ of interest doses of: Liver≧600 cGy, Kidney≧300 cGy, or marrow≧40 cGy, or Grade≧3 renal, audiometry or hematological or other toxicity according to the Common Toxicity Criteria Version 2.0 (CTC v2.0) they will continue to receive their assigned dosage monthly for up to 12 months. The following will be performed at each visit:

History/Physical Exam

CBC, platelets

Signs and Symptoms

CTorMRI

BUN and Creatinine

Tumor marker titer

Liver Function

Audiometry

ECG

SPECT/planar imaging

Dosing

### SPECT/Planar Imaging

Single Photon Emission Computed Tomographic (SPECT)/Planar imaging will be used to determine the activity, absorbed dose and tumor volume. Data analyses are provided in Siegel et al.. Each SPECT procedure consists of three imaging sessions for each patient. Imaging sessions will be performed immediately after radioactive drug infusion and then four days (one half-life) after drug administration. Blood samples will also be taken a multiple time points to determine the pharmacokinetics of radioactively-labeled platinum compounds at each SPECT scan.

### REFERENCES

Amorino G P, Freeman M L, Carbone D P, et al. Radiopotentiation by the oral platinum agent, JM216:role of repair inhibition. Int J Radiat Oncol Biol Phys 44(2):399-405, 1999.

Anand D, Wolf W. A new, semi-automated system for the micro-scale synthesis of [195mPt]cisplatin suitable for clinical studies. Int J Rad Appl Instrum 43(6):809-814, 1992.

Anderson H, Wagstaff, Crowther D, et al. Comparative toxicity of cisplatin, carboplatin (CBDCA) and iproplatin (CHIP) in combination with cyclophosphamide in patients with advanced epithelial ovarian cancer. European J Cancer Clin Oncol 24(9):1471-1479, 1988.

Areberg J., Johnsson A, Wennerberg J. In vitro toxicity of ¹⁹¹Pt-labeled cisplatin to a human cervical carcinoma cell line (ME-180). Int J Radiation Oncol Biol Phys 46(5):1275-1280, 2000

Areberg J, Wennerberg J, Johnsson A, et al. Antitumor effect of radioactive cisplatin (191 Pt) on nude mice. Int J Radiat Oncol Biol Phys 49(3):827-832, 2001.

Baer J, Harrison R, McAuliffe C A, et al. Microscale synthesis of anti-tumor platinum compounds labeled with 191 Pt. Int J Appl Radiat Isot 36(3):181-184, 1985.

Barot H A, Laverick M, Nias A H. The radiomimetic properties of a platinum drug. Br J Radiol 58(685):51-62, 1985.

Bates P I, Sharma H L, Murrer B A, et al. The synthesis of 191 Pt labeled JM216, an orally active platinum anti-tumour agent. Appl Radiat Isot 48(4):469-476, 1997.

Beale P, Raynaud F, Hanwell J, et al. Phase I study of oral JM216 given twice daily. Cancer Chemother Pharmacol 42(2):142-148.

Bramwell V H, Crowther D, O'Malley S, et al. Activity of JM9 in advanced ovarian cancer: a phase I-II trial. Cancer Treat Rep 69(4):409-416, 1985.

Bunn P A, et al. Carboplatin (JM-8): Current perspectives and future directions, Philadelphia, 1990, W B Saunders.

Chawla S P, Yap B S, Tenney D M, et al. Phase I study of weekly-administered iproplatin [cis-dichloro-trans-dihydroxy-bis-isopropylamine platin (chip, JM9)]. Invest New Drugs 6(4):311-317, 1988.

Cheung Y W, Cradock J C, Vishnuvajjala B R, et al. Stability of cisplatin, iproplatin, carboplatin and tetraplatin in commonly used intravenous solutions. Am J Hosp Pharm 44(1):124-130, 1987.

Clark D L, Andrews P A, Smith D D, et al. Predictive value of preclinical toxicology studie sfor platinum anticancer drugs. Clin Cancer Res 595):1161-1167, 1999.

Dowell J A, Sancho A R, Anand D, et al. Noninvasive measurements for studying the tumoral pharmacokinetics of platinum anticancer drugs in solid tumors. Adv Drug Deliv Rev 4(1):111-126, 2000.

Douple E B, Richmond R C, O'Hara J A, et al. Carboplatin as a potentiator of radiation therapy. Cancer treat Rev 12(A): 111 -124,1985.

Fisher D S, Knobf M T, Durivage H J, eds: The Cancer Chemotherapy Handbook, St. Louis, 1997, Mosby.

Harrison R, McAuliffe C A, Zaki A, et al. A comparative study of the distribution in the male rat of platinum-labelled cis-dichlorodiammine platinum (II), cis-trans-dichlorodihydroxy-bis-(isopropylamine) platinum (I), and cis-dichloro-bis-cyclopropylamine platinum (II). Cancer Chemother Pharmacolo 10(2):90-95, 1983.

Holford J, Beale P J, Boxall F E, et al. Mechanisms of drug resistance to the platinum complex ZD0473 in ovarian cancer cell lines. Eur J Cancer 36(15):1984-1990, 2000

Howell R W, Kassis A I, Adelstein J, et al. Radiotoxicity of platinum-195m-labeled trans-Platinum (II) in mammalian cells. Radiation Research 140:55-62, 1994.

Judson I. New developments and approaches in the platinum arena. Drugs 2000; S4:29, 2000.

Kelland L R, Sharp S Y, O'Neill C F, et al. Mini-review: discovery and development of platinum complexes designed to circumvent cisplatin resistance. J Inorg Biochem 77(1-2):111-115,1999.

Kelland L R. An update on satraplatin: the first orally available platinum anticancer drug. Expert Opin Investig Drugs 9(6):1373-1382, 2000.

Kurata T, Tamura T, Sasaki Y, et al. Pharmacokinetic and pharmacodynamic analysis of bis-aceto-ammine-dichloro-cyclohexylamine-platinum(IV) (JM216) administered once a day for five consecutive days:a phase I study. Jpn J Clin Oncol 30(9):377-384, 2000.

McKeage M J, Mistry P, Ward J, et al. A phase I and pharmacology study of an oral platinum complex, JM216: dose-dependent pharmacokinetics with single-dose administration. Cancer Chemother Pharmacol 36(6):451-458, 1995.

O'Dwyer P J, Stevenson J P, Johnson S W. Clinical pharmacokinetics and administration of established platinum drugs. Drugs 2000: 59(S4):19-27, 2000.

Order SE. Radioactive cisplatinum in the treatment of cancer. (U.S. Pat. No. 6,074,626).

Owens S E, Thatcher N, Sharma H, et al. In vivo distribution studies of radioactively labelled platinum complexes; cis-dichlorodiammine platinum (II), cis-trans-dichlorodihydroxy-bis-(isopropylamine) platinum (IV) cis-dichloro-bis-cyclopropylamine platinum (II), and cis-diammine 1,1-cyclobutanedicarboxylate platinum (II) in patients with malignant disease, using a gamma camera. Cancer Chemother Pharmacol 14:253-257,1985.

Reed E. Platinum analogs. In: Devita, Heliman and Rosenberg, eds. Cancer, Principles and Practice of Oncology 4^{th} edition. Philadelphia: J B Lippincott & Company, 1993, 390-400.

Ribaud P, Gouveia J, Misset J L, et al. Phase I study of cis-dichloro-trans-dihydroxy-bis(isopropylamine)platinum IV (CHIP). Oncology 43(2):78-82, 1986.

Rixe O, Ortuzar W, Alvarez M, et al. Oxaliplatin, Tetraplatin, cisplatin and carboplatin: spectrum of activity in drug-resistant cell lines and in the cell lines of the National Cancer Institute's Anticancer Drug Screen Panel. Biochem Pharmacol 52(12):1855-65, 1996.

Sessa C, Minoia C, Ronchi A, et al. Phase I clinical and pharmacokinetic study of the oral platinum analog JM216 given daily for 14 days. Ann Oncol 9(12):1315-1322, 1998.

Sharma H, Thatcher N, Baer J, et al. Blood clearance of radioactively labelled cis-diammine 1,1-cyclobutane dicarboxylate platinum(lI) (CBDCA) in cancer patients. Cancer Chemother Pharmacol 11:5-7, 1983.

Skov K, MacPhail S. Interaction of platinum drugs with clinically relevant x-ray doses in mammalian cells: a comparison of cisplatin, carboplatin, iproplatin and tetraplatin. Int J Radiat Oncol Biol Phys 20(2):221-225, 1991.

Tanaka M, Kuwahara E, Shimizu T, et al. Rectal absorption of [Bis(acetato) ammine dichloro (cyclohexylamine) platinum (IV)] (BMS-182751), a new anti-tumor agent, in rats. Biol Pharm Bull 22(5):521-526, 1999.

Thatcher N, Sharma H, Harrison R, et al. Blood clearance of three radioactively labeled platinum complexes: cis-dichlorodiammine platinum II, cis, trans-dichlorodihydroxy-bis-(isopropylamine) platinum IV, and cis-dichloro-bis-cyclopropylamine platinum II, in patients with malignant disease. Cancer Chemother Pharmacol 9(1): 13-16, 1982.

Trask C, Silverstone A, Ash C M, et al. A randomized trial of carboplatin versus iproplatin in untreated advanced ovarian cancer. J Clin Oncology 9(7):1131-1137, 1991.

TABLE 1
Common and chemical drug names

Common Name

| | |
|---|---|
| Carboplatin | Carboplatinum |
| | Paraplatin |
| | CBDCA |
| | JM-8 |
| | NSC-241240 |
| | CAS# 41575-94-4 |
| | cis-(1,1-Cyclobutanedicarboxylato)diamineplatinum (II) |
| | cis-Diamine(1,1-cyclobutanedicarboxylato)platinum (II) |
| | Platinum, diamine(1,1-cyclobutanedicarboxylato(2-)-O, |
| | O′)-,(SP-4-2) |
| | 1,1-Cyclobutanedicarboxylate diamine platinum (II) |
| | 1,1-Cyclobutanedicarboxylatodiammineplatinum (II) |
| JM216 | Satraplatin |
| | BMS-182751 |
| | BMY-45594 |
| | CAS# 129580-63-8 |
| | bis-acetato-ammine-dichloro-cyclohexylamine platinum |
| | (IV)) |
| Iproplatin | JM9 |
| | CHIP |
| | NSC-256927 |
| | CAS# 83291-20-7 |
| | Cis,trans-PtCl2(OH)2(isopropylamine)(IV) |
| | Cis-dichloro,trans-dihydroxybis-isopropylamine platinum |
| | (IV) |

TABLE 2
Drug development status

| Drug | Source | Stage of testing | Indications for use |
|---|---|---|---|
| Carboplatin | Bristol-Myers | FDA approved | Neuroblastoma, |
| | Squibb | | refractory leukemia, |
| | | | cancers of the ovary, |
| | | | bladder, brain, breast, |
| | | | testes, lung, cervix, |
| | | | endometrium, head and |
| | | | neck. |
| JM216 | Bristol-Myers | Phase III | Prostate, small cell lung, |
| | Squibb | | ovarian cancers |
| Iproplatin | | Phase I, II | Testicular and ovarian |
| | | | cancer |
| | | | Solid tumors |

TABLE 3
Conventional drug dosing regimens

| | | | | Refer- |
|---|---|---|---|---|
| Drug | Disease | Dose regimen | Administration | ences |
| Carboplatin | Solid | 360-400 mg/m2 | i.v. saline or 5% | Fischer |
| | tumors | every 4 weeks | dextrose over | 1997 |
| | | | 15-30 min | |
| JM216 | Solid | 120 mg/m2 × 5 | p.o. gelatin | McKeage |
| | tumors | days (60-700 | capsules | 1995 |
| | | tested | | |
| | Solid | 40-45 mg/m2 × | p.o. | Sessa |
| | tumors | 14 days every | | 1998 |
| | | 5 weeks | | |
| | Solid | 100 mg/m2 × 5 | p.o. | Kurata |
| | tumors | days every five | | 2000 |
| | | weeks | | |
| Iproplatin | Ovarian | 180-350 mg/m² | i.v. in 1 L saline | Bramwell |
| | | once every 3 | over 1 hour | 1985 |
| | | weeks | | |
| | Solid | 20-45 mg/m² × 5 | i.v. in saline | Ribaud |
| | tumors | days every 6 | over 1 hour | 1986 |
| | | weeks | | |
| | Solid | 40-95 mg/m² | i.v | Chawla |
| | tumors | once per week × | | 1998 |
| | | 4 weeks | | |
| | Ovarian | 240 mg/m2 | i.v | Anderson |
| | | | | 1988 |
| | Ovarian | 225-300 mg/m2 | i.v. | Trask |
| | | every 2 weeks | | 1991 |

TABLE 4
Radiolabeled drug studies

| | | Radiation | |
|---|---|---|---|
| | Isotopic forms of Pt | enhancer | |
| Drug | produced | in vitro? | Human studies |
| Carbo- | 191, 193 m | Yes | Biodistribution |
| platin | (Owens 1985; Baer | (Amorino | (Owens |
| | 1985) | 1999;Skov 1991; | 1985;Sharma 1983) |
| | | Douple 1985) | |
| JM216 | 191 | Yes | N/A |
| | (Bates 1997) | (Amorino 1999) | |
| Iproplatin | 191, 193 m | Yes | Biodistribution |
| | (Owens 1985; Baer | (Barot 1985;Skov | (Owens 1985 |
| | 1985) | 1991) | Thatcher 1982) |

1. A method of treating cancer comprising administering to a living being a therapeutically effective amount of composition comprising a radioactive platinum compound.

2. The method of claim 1 additionally comprising the step of selecting the composition to additionally comprise a physiologically acceptable carrier.

3. The method of claim 1 wherein the composition is administered intra-arterially.

4. The method of claim 1 wherein the composition is administered orally.

5. The method of claim 1 wherein the composition is administered in intra-peritoneally.

6. The method of claim 1 wherein the composition is administered intra-thecally.

7. The method of claim 1 wherein the composition is administered intra-tumorally.

8. The method of claim 1 wherein the radioactive platinum compound is selected from the group consisting of
(a) ¹⁹¹Pt-labeled 1,1-Cyclobutanedicarboxylate diamine platinum (II);
(b) ^{193m}Pt-labeled 1,1-Cyclobutanedicarboxylate diamine platinum (II);
(c) ^{195m}Pt-labeled 1,1-Cyclobutanedicarboxylate diamine platinum (II);
(d) ¹⁹¹Pt-labeled Cis-dichloro,trans-dihydroxybis-isopropylamine, platinum (IV);
(e) ^{193m}Pt-labeled Cis-dichloro,trans-dihydroxybis-isopropylamine, platinum (IV);
(f) ^{195m}Pt-labeled Cis-dichloro,trans-dihydroxybis-isopropylamine, platinum (IV);
(g) ¹⁹¹Pt-labeled bis-acetato-ammine-dicholoro-cyclohexylamine platinum (IV);
(h) ^{193m}Pt-labeled bis-acetato-ammine-dicholoro-cyclohexylamine platinum (IV); and
(i) ¹⁹⁵mPt-labeled bis-acetato-ammine-dicholoro-cyclohexylamine platinum (IV).

9. A pharmaceutical composition for treating cancer comprising a therapeutically effective amount of a radioactive platinum compound and a carrier material.

10. The composition of claim 9 wherein the radioactive platinum compound is selected from the group consisting of
(a) ¹⁹¹Pt-labeled 1,1-Cyclobutanedicarboxylate diamine platinum (II);
(b) ^{193m}Pt-labeled 1,1-Cyclobutanedicarboxylate diamine platinum (II);
(c) ^{195m}Pt-labeled 1,1-Cyclobutanedicarboxylate diamine platinum (II);
(d) ¹⁹¹Pt-labeled Cis-dichloro,trans-dihydroxybis-isopropylamine, platinum (IV);
(e) ^{193m}Pt-labeled Cis-dichloro,trans-dihydroxybis-isopropylamine, platinum (IV);
(f) ^{195m}Pt-labeled Cis-dichloro,trans-dihydroxybis-isopropylamine, platinum (IV);
(g) ¹⁹¹Pt-labeled bis-acetato-ammine-dicholoro-cyclohexylamine platinum (IV);
(h) ^{193m}Pt-labeled bis-acetato-ammine-dicholoro-cyclohexylamine platinum (IV); and
(i) ^{195m}Pt-labeled bis-acetato-ammine-dicholoro-cyclohexylamine platinum (IV).
